(19) United States
(12) Patent Application Publication
(10) Pub. No.: US 2005/0221375 A1
(43) Pub. Date: Oct. 6, 2005

(54) Multiplex sequencing method

(76) Inventor: Rudolf Rigler, St-Sulpice (CH)

Correspondence Address: FULBRIGHT & JAWORSKI, LLP, 666 FIFTH AVE, NEW YORK, NY, 10103-3198, US

(21) Appl. No.: 11/139,080

(22) Filed: May 26, 2005

Related U.S. Application Data
(63) Continuation of application No. 10/311,439, filed on Dec. 17, 2002, now abandoned.

(30) Foreign Application Priority Data
Jun. 30, 2000 (DE) 100 31 842.8
Jun. 29, 2001 (WO) PCT/EP01/07462

(51) Int. Cl. C12Q 1/68

(52) U.S. Cl. 435/6

(57) ABSTRACT

The invention relates to a method and a device for multiplex sequencing of nucleic acid molecules immobilized on a support.

The invention relates to a method and a device for multiplex sequencing of nucleic acid molecules immobilized on a support.

Sequencing of the human genome which consists of approx. 3×10⁹ bases, or of the genome of other organisms and the determination and comparison of individual sequence variants require the provision of sequencing methods which firstly are fast and secondly can be employed routinely and cost-effectively. Although large efforts have been made in order to accelerate familiar sequencing methods, for example the enzymic chain termination method according to Sanger et al. (Proc. Natl. Acad. Sci. USA 74 (1977) 5463), in particular by automation (Adams et al., Automated DNA Sequencing and Analysis (1994), New York, Academic Press), currently only up to 2 000 bases per day can be determined using an automated sequencer.

Over recent years, new approaches to overcome the limitations of conventional sequencing methods have been developed, inter alia sequencing by scanning tunneling microscopy (Lindsay and Phillip, Gen. Anal. Tech Appl. 8 (1991), 8-13), by highly parallel capillary electrophoresis (Huang et al., Anal. Chem. 64 (1992), 2149-2154; Kambara and Takahashi, Nature 361 (1993), 565-566), by oligonucleotide hybridization (Drmanac et al., Genomics 4 (1989), 114-128; Khrapko et al., FEBS Let. 256 (1989), 118-122; Maskos and Southern, Nucleic Acids Res. 20 (1992), 1675-1678 and 1679-1684) and by matrix-assisted laser desorption/ionization mass spectroscopy (Hillenkamp et al., Anal. Chem. 63 (1991), 1193A-1203A).

Another approach is single-molecule sequencing (Dorre et al., Bioimaging 5 (1997), 139-152), in which the sequence of nucleic acids is carried out by progressive enzymic degradation of fluorescently labeled single-stranded DNA molecules and by detection of the sequentially released monomeric molecules in a microstructure channel. This method has the advantage of only a single molecule of the target nucleic acid being sufficient for carrying out a sequence determination.

Although application of the abovementioned methods has already resulted in considerable progress, there is a great need for further improvements. The object on which the present invention is based was therefore to provide a method for sequencing nucleic acids, which represents a further improvement compared to the prior art and which makes possible parallel determination of individual nucleic acid molecules in a multiplex format.

This object is achieved by a method for sequencing nucleic acids, which comprises the following steps:
(a) providing a support with a multiplicity of nucleic acid molecules immobilized thereon, said nucleic acid molecules carrying a plurality of fluorescent labeling groups,
(b) progressively removing by cleavage individual nucleotide building blocks from said immobilized nucleic acid molecules and
(c) determining simultaneously the base sequence of a plurality of nucleic acid molecules owing to the time-dependent change in fluorescence of said nucleic acid molecules or/and the nucleotide building blocks removed by cleavage, which change is caused by removing said nucleotide building blocks by cleavage.

The method of the invention is a support-based multiplex sequencing method in which a multiplicity of immobilized nucleic acid molecules is studied at the same time. The support used for said method may be any planar or structured support which is suitable for immobilizing nucleic acid molecules. Examples of suitable support materials are glass, plastic, metals or semimetals such as, for example, silicon, metal oxides such as silicon oxide, etc. In principle, the support may also have any design, as long as a reaction space can be formed which makes possible the progressive removal by cleavage of individual nucleotide building blocks from the nucleic acids immobilized on the support in a liquid reaction mixture.

The nucleic acid molecules are preferably immobilized on the support via their 5′ or 3′ ends and may be present in single-stranded or double-stranded form. In the case of double-stranded molecules, it must be ensured that labeled nucleotide building blocks can be removed by cleavage only from one single strand. The nucleic acid molecules can bind to the support via covalent or noncovalent interactions. For example, high-affinity interactions between the partners of a specific binding pair, for example biotin/streptavidin or avidin, haptene/anti-haptene antibody, sugar/lectin, etc., can mediate binding of the polynucleotides to the support. Thus it is possible to couple biotinylated nucleic acid molecules to streptavidin-coated supports. Alternatively, the nucleic acid molecules may also be bound to the support via adsorption. Thus nucleic acid molecules modified by incorporation of alkane thiol groups, may bind to metallic supports, e.g. supports made of gold. Yet another alternative is covalent immobilization in which it is possible to mediate polynucleotide binding via reactive silane groups on a silica surface.

A plurality of nucleic acid molecules intended for sequencing is bound to a single support. Preferably at least 100, particularly preferably at least 1 000, and particularly preferably at least 10 000 and up to more than 10⁶, nucleic acid molecules are bound to said support. The nucleic acid fragments bound are preferably from 200 to 2 000 nucleotides in length, particularly preferably 400 to 1 000 nucleotides. The nucleic acid molecules bound to the support, for example DNA molecules or RNA molecules, contain a plurality of fluorescent labeling groups, with preferably at least 50%, particularly preferably at least 70% and most preferably essentially all, for example at least 90%, of the nucleotide building blocks of a single base type carrying a fluorescent labeling group. Nucleic acids labeled in this way may be generated by enzymic primer extension on a nucleic acid template by using a suitable polymerase, for example a DNA polymerase such as, for example, Taq polymerase, a thermostable DNA polymerase from Thermococcus gorgonarius or other thermostable organisms (Hopfner et al., PNAS USA 96 (1999), 3600-3605), or a mutated Taq polymerase (Patel and Loeb, PNAS USA 97 (2000), 5095-5100), by using fluorescently labeled nucleotide building blocks.

It is also possible to prepare the labeled nucleic acid molecules by amplification reactions, for example PCR. Thus an asymmetric PCR produces amplification products in which only one strand contains fluorescent labels. Such asymmetric amplification products can be sequenced in double-stranded form. Symmetric PCR produces nucleic acid fragments in which both strands are fluorescently labeled. These two fluorescently labeled strands can be separated and immobilized separately in single-stranded form so that it is possible to determine the sequence of one or both complementary strands separately. Alternatively, one of the two strands can be modified on the 3′ end, for example by incorporating a PNA link, such that monomeric building blocks can no longer be removed by cleavage. In this case, double-strand sequencing is possible.

Preferably, essentially all nucleotide building blocks of at least two base types, for example two, three or four base types, carry a fluorescent label, each base type conveniently carrying a different fluorescent labeling group. If the nucleic acid molecules have not been labeled completely, it is nevertheless possible to determine the sequence completely by parallel sequencing of a plurality of molecules.

The nucleic acid template whose sequence is to be determined may be selected, for example, from DNA templates such as genomic DNA fragments, cDNA molecules, plasmids, etc., or else from RNA templates such as mRNA molecules.

The fluorescent labeling groups may be selected from known fluorescent labeling groups used for labeling biopolymers, for example nucleic acids, such as, for example, fluorescein, rhodamine, phycoerythrin, Cy3, Cy5 or derivatives therefrom, etc.

The method of the invention is based on fluorescent labeling groups incorporated in nucleic acid strands interacting with neighboring groups, for example with chemical groups of the nucleic acids, in particular nucleobases such as, for example, G, or/and neighboring fluorescent labeling groups, and these interactions leading to a change in fluorescence, in particular in fluorescence intensity, compared to the fluorescent labeling groups in “isolated” form, owing to quenching processes or/and energy transfer processes. The removal by cleavage of individual nucleotide building blocks alters the overall fluorescence, for example the fluorescence intensity of an immobilized nucleic acid strand, and this change is a function of the removal by cleavage of individual nucleotide building blocks, i.e. a function of time. This time-dependent change in fluorescence may be recorded in parallel for a multiplicity of nucleic acid molecules and correlated with the base sequence of the individual nucleic acid strands. Preference is given to using those fluorescent labeling groups which, when incorporated in the nucleic acid strand, are, at least partially, quenched so that the fluorescence intensity is increased after the nucleotide building block containing the labeling group or a neighboring building block causing quenching has been removed by cleavage.

The sequencing reaction of the method of the invention comprises progressively removing by cleavage individual nucleotide building blocks from the immobilized nucleic acid molecules. Preferably the removal by cleavage is carried out enzymatically using an exonuclease, it being possible to use single-strand or double-strand exonucleases which cleave in the 5′→3′ direction or 3′→5′ direction, depending on the type of immobilization of the nucleic acid strands on the support. Exonucleases which are particularly preferably used are T7 DNA polymerase, E. coli exonuclease I and E. coli exonuclease III.

During the progressive removal by cleavage of individual nucleotide building blocks, it is possible to measure a change in fluorescence intensity of the immobilized nucleic acid strand or/and the nucleotide building block removed by cleavage, owing to quenching processes or energy transfer processes. This change in fluorescence intensity with time depends on the base sequence of the nucleic acid strand studied and can therefore be correlated with the sequence. The complete sequence of a nucleic acid strand is usually determined by generating, preferably by enzymic primer extension as described above, and immobilizing on the support a plurality of nucleic acid strands, labeled on different bases, for example A, G, C and T, or combinations of two different bases, it being possible for the immobilization to take place at random locations or else at specific locations on the support. It is possible, where appropriate, to attach to the nucleic acid strand to be studied also a “sequence identifier”, i.e. a labeled nucleic acid of known sequence, for example by enzymic reaction using ligase or/and terminal transferase, so that at the start of sequencing initially a known fluorescence pattern and only thereafter the fluorescence pattern corresponding to the unknown sequence to be studied is obtained. The total number of nucleic acid strands immobilized on a single support is preferably 10³ to 10⁶.

In order to accelerate the removal of cleaved nucleotide building blocks from the immobilized nucleotide strands, preferably a convectional flow away from the support is generated in the reaction space. The flow rate used herein may be in the range from 1 to 10 mm/s.

The detection preferably comprises a multipoint fluorescence excitation by lasers, for example a dot matrix of laser dots generated via diffraction optics or a quantum well laser. The fluorescence emission, generated by excitation of a plurality of nucleic acid strands may be generated by a detector matrix which comprises, for example, an electronic detector matrix, for example a CCD camera or an avalanche photodiode matrix. The detection may be carried out in such a way that fluorescence excitation and detection are carried out in parallel on all nucleic acid strands studied. A possible alternative of this is to study in several steps in each case a portion of the nucleic acid strands by using a submatrix of laser dots and detectors preferably by using a high-speed scanning procedure.

The invention further relates to a support for sequencing nucleic acids, which comprises a multiplicity of nucleic acid molecules immobilized thereon, said nucleic acid molecules being in single-stranded form and carrying a plurality of fluorescent labeling groups.

The invention still further relates to a device for sequencing nucleic acids, comprising
(a) a support as specified above,
(b) a reaction space for progressively removing by cleavage individual nucleotide building blocks from said immobilized nucleic acid molecules and
(c) means for determining simultaneously the base sequence of a plurality of nucleic acid molecules owing to the time-dependent change in fluorescence of said nucleic acid molecules or/and the nucleotide building blocks removed by cleavage, which change is caused by removing said nucleotide building blocks by cleavage.

The method of the invention may be used, for example, for the analysis of genomes and transcriptomes or for differential analyses, for example studies with respect to the difference in the genome or transcriptome of individual species or organisms of a species.

Furthermore, the present invention is intended to be illustrated by the following figures in which:

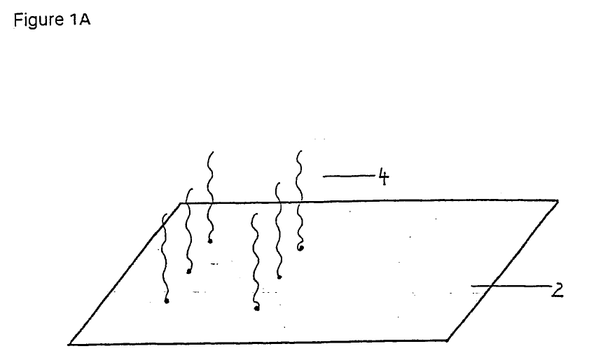

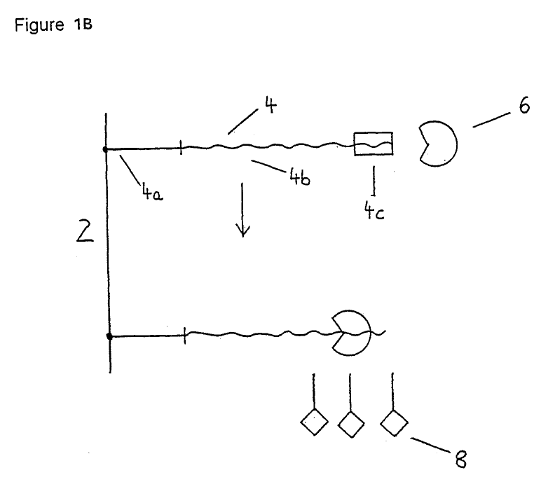

FIG. 1:
(A) shows the diagrammatic representation of a support of the invention (2) having a multiplicity of single-stranded nucleic acid molecules immobilized thereto (4). A support with an area of from 1 to 2 cm² may contain, for example, up to 10⁶ nucleic acid strands.
(B) shows the nucleic acid molecules (4) immobilized to the support (2), which may comprise a 5′-biotinylated primer (or a primer provided with a different solid phase binding group) (4a), a fluorescently labeled section to be sequenced (4b) and, where appropriate, a fluorescently labeled sequence identifier (4c). Single nucleotide building blocks (8) are continuously removed by cleavage due to the action of an exonuclease (6). While the nucleotide building blocks incorporated into the nucleic acid strand exhibit only little fluorescence, if at all, due to quenching processes, fluorescence is increased after the cleavage.

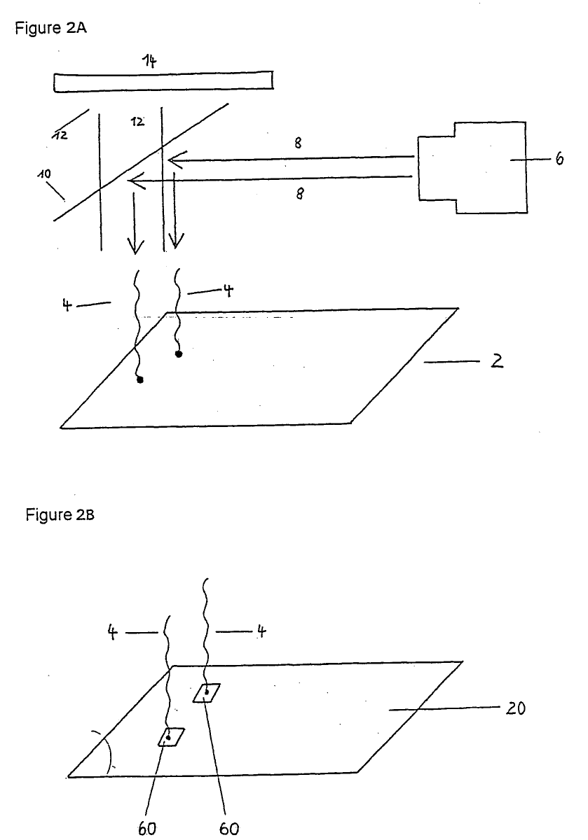

FIG. 2:
(A) depicts a first embodiment of the invention, wherein the nucleic acid molecules (4) immobilized on the support (2) are irradiated by a laser (6) with excitation light (8) which is directed to the individual immobilized nucleic acid strands by a diffraction optics element (10). The fluorescence emission light (12) is recorded by a detector matrix (14), for example a CCD camera.
(B) In a further embodiment of the invention, a support (20) with a quantum well laser integrated therein (60) is used.

1-22. (canceled)

23. A method for sequencing nucleic acids, which comprises the following steps:
(a) providing a support with a multiplicity of nucleic acid molecules immobilized thereon, said nucleic acid molecules carrying a plurality of fluorescent labeling groups,
(b) progressively removing by cleavage individual nucleotide building blocks from said immobilized nucleic acid molecules and
(c) determining-simultaneously the base sequence of a plurality of nucleic acid molecules owing to the time-dependent change in fluorescence of said nucleic acid molecules or/and the nucleotide building blocks removed by cleavage, which change is caused by removing said nucleotide building blocks by cleavage.

24. The method as claimed in claim 23, wherein a planar support is used.

25. The method as claimed in claim 23, wherein a structured support is used.

26. The method as claimed in claim 23, wherein the nucleic acid molecules are immobilized on the support via their 5′ or 3′ ends.

27. The method as claimed in claim 23, wherein the nucleic acid molecules are immobilized on the support in single-stranded form.

28. The method as claimed in claim 23 wherein the nucleic acid molecules are immobilized on the support in double-stranded form, it being possible to remove labeled nucleotide building blocks by cleavage only from one single strand.

29. The method as claimed in claim 23, wherein the nucleic acid molecules are labeled so that at least 50% of all nucleotide building blocks of a single base type carry a fluorescent labeling group.

30. The method as claimed in claim 29, wherein essentially all nucleotide building blocks of a single base type carry a fluorescent labeling group.

31. The method as claimed in claim 23, wherein individual nucleotide building blocks are removed by cleavage by an exonuclease.

32. The method as claimed in claim 31, wherein T7 DNA polymerase, E. coli exonuclease I or E. coli exonuclese III is used.

33. The method as claimed in claim 23, wherein determining the base sequence comprises a multipoint fluorescence excitation by lasers.

34. The method as claimed in claim 23, wherein determining the base sequence comprises detection of the fluorescence emission of a plurality of nucleic acid strands via a detection matrix.

35. The method as claimed in claim 34, wherein a CCD camera or an avalanche photodiode matrix is used.

36. The method as claimed in claim 34, wherein fluorescence excitation and fluorescence detection are carried out in parallel on all nucleic acid strands studied.

37. The method as claimed in claim 34, wherein fluorescence excitation and fluorescence detection are carried out in several steps, in each case on a portion of the nucleic acid strands studied, using a submatrix of laser dots and detectors.

38. The method as claimed in claim 34, wherein a convectional flow away from the support is generated during determination.

39. The method as claimed in claim 34, wherein the fluorescent labeling groups are, at least partially, quenched when incorporated into the nucleic acid strands and that the fluorescence intensity is increased after removal by cleavage.

40. A support for sequencing nucleic acids, which comprises a multiplicity of nucleic acid molecules immobilized thereon, said nucleic acid molecules being in single-stranded form and carrying a plurality of fluorescent labeling groups.

41. The support as claimed in claim 40, wherein the nucleic acid molecules are labeled so that at least 500 of all nucleotide building blocks of a single base type carry a fluorescent labeling group.

42. The support as claimed in claim 40, wherein the nucleic acid molecules are from 200 to 2000 nucleotides in length.

43. A device for sequencing nucleic acids, comprising
(a) a support as claimed in claim 40
(b) a reaction space for progressively removing by cleavage individual nucleotide building blocks from said immobilized nucleic acid molecules and
(c) means for determining simultaneously the base sequence of a plurality of nucleic acid molecules owing to the time-dependent change in fluorescence of said nucleic acid molecules or/and the nucleotide building blocks removed by cleavage, which change is caused by removing said nucleotide building blocks by cleavage.

44. The support as claimed in claim 41, wherein the nucleic acid-molecules are from 200 to 2,000 nucleotides in length.

45. A method for sequencing nucleic acids, which comprises the following steps:
(a) providing a support with a multiplicity of nucleic acid molecules immobilized thereon, wherein said immobilization is facilitated by a hapten/anti-hapten antibody complex, wherein said nucleic acid molecules are immobilized on the support via their 3′ ends, and said nucleic acid molecules carrying a plurality of fluorescent labeling groups,
(b) progressively removing by cleavage individual nucleotide building blocks from said immobilized nucleic acid molecules and
(c) determining-simultaneously the base sequence of a plurality of nucleic acid molecules owing to the time-dependent change in fluorescence of said nucleic acid molecules or/and the nucleotide building blocks removed by cleavage, which change is caused by removing said nucleotide building blocks by cleavage.
